(19) United States
(12) Patent Application Publication
(10) Pub. No.: US 2025/0145502 A1
(43) Pub. Date: May 8, 2025

(54) WATER TREATMENT OF SMALL WATER RESERVOIRS WITH LIMITED INFRASTRUCTURE AND NO ADDITION OF CHEMICALS

(71) Applicant: Atlantium Technologies Ltd., Beit-Shemesh (IL)

(72) Inventors: Ytzhak ROZENBERG, Caesarea (IL); Amichai FELDER, Givatayim (IL); Rachel GOTTLIEB, Jerusalem (IL); Itay BELFER, Modi`in (IL); Assaf LOWENTHAL, Rehovot (IL)

(73) Assignee: Atlantium Technologies Ltd., Beit-Shemesh (IL)

(21) Appl. No.: 18/832,583

(22) PCT Filed: Dec. 18, 2022
(86) PCT No.: PCT/IL2022/051345

§ 371 Date: Jul. 24, 2024

Related U.S. Application Data
(60) Provisional application No. 63/303,266, filed on Jan. 26, 2022.

(51) Int. Cl. C02F 1/32; A61L 2/10; A61L 2/24; A61L 2/26; C02F 1/00; C02F 103/42

(52) U.S. Cl. CPC C02F 1/32; A61L 2/10; A61L 2/24; A61L 2/26; C02F 1/001; C02F 1/008; A61L 2202/11; A61L 2202/14; A61L 2202/15; C02F 2103/42; C02F 2201/3222; C02F 2209/03; C02F 2301/046; C02F 2303/04; C02F 2303/16

(57) ABSTRACT

Water treatment systems and methods are provided, for treating a reservoir without addition of chemicals. Such requirements arise mainly for relatively small reservoirs with limited filtering infrastructure, such as private pools. Ultraviolet (UV) treatment is employed, in addition or possibly in place of filtering, to overcome infrastructural limitations on water throughput, such as filtering and/or pumping capacity. UV may be used over longer periods and/or with respect to higher throughputs, to reduce bacterial growth rates more significantly than filtering, and thereby prevent bacterial growth in the reservoir. UV treatment may be applied according to a pre-defined temporal pattern selected to prevent biomass growth in the reservoir and optionally be updated with respect to water quality measurements.

### BACKGROUND OF THE INVENTION

### 1. Technical Field

The present invention relates to the field of water treatment, and more particularly, to ultraviolet (UV) treatment of small reservoirs.

### 2. Discussion of Related Art

Small reservoirs, such as private pools, are typically not treated with chemicals and typically have limited filtering infrastructure, which pose the risk of bacterial contamination.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a water treatment system for treating a reservoir without addition of chemicals, the water treatment system comprising: a filtering unit, an ultraviolet (UV) treatment unit, a pumping unit configured to circulate water from the reservoir through the filtering unit and back to the reservoir, and configured to circulate water from the reservoir through the UV treatment unit and back to the reservoir, and a control unit configured to operate the pumping unit, the filtering unit and the UV treatment unit at a pre-defined temporal pattern selected to prevent biomass growth in the reservoir.

One aspect of the present invention provides a water treatment method of treating a reservoir without addition of chemicals, the water treatment method comprising: circulating and treating with ultraviolet (UV) radiation water from the reservoir, in addition to filtering at most a portion of the circulated water, wherein the UV water treatment is carried out according to a pre-defined temporal pattern selected to prevent biomass growth in the reservoir.

One aspect of the present invention provides a computer program product comprising a non-transitory computer readable storage medium having computer readable program embodied therewith, the computer readable program comprising: computer readable program configured to receive measurements of a biomass level in water of a reservoir that is treated by ultraviolet (UV) radiation without addition of chemicals, and update a pre-defined temporal pattern of the UV treatment if the measured biomass level exceeds a pre-defined threshold.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A-1C are high-level schematic illustrations of water treatment systems, according to some embodiments of the invention.

FIG. 1D provides simulation results depicting the expected bacterial concentration over time using water treatment systems, according to some embodiments of the invention.

FIGS. 1E and 1F are high-level schematic illustrations of pump operation according to water contamination level, according to some embodiments of the invention.

FIG. 2A is a high-level schematic illustration of prior art water treatment of private pools.

FIGS. 2B and 2C provide simulation results depicting the expected bacterial concentration over time for two filtering durations in the prior art.

FIG. 3B is a high-level schematic illustration of the efficiency of UV water treatment, according to some embodiments of the invention, as contrasted with prior art use of filtering alone, illustrated schematically in FIG. 3A.

FIG. 4 is a high-level block diagram of exemplary controllers, which may be used with embodiments of the present invention.

FIG. 5 is a high-level flowchart illustrating water treatment methods of treating a reservoir without addition of chemicals, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Unless specifically stated otherwise, as apparent from the following discussions, it is appreciated that throughout the specification discussions utilizing terms such as “processing”, “computing”, “calculating”, “determining”, “enhancing”, “deriving” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the present invention provide efficient and economical methods and mechanisms for water treatment and thereby provide improvements to the technological field of preventing bacterial contamination of small water reservoirs. Water treatment systems and methods are provided, for treating a reservoir without addition of chemicals. Such requirements arise mainly for relatively small reservoirs with limited filtering infrastructure, such as private pools. Ultraviolet (UV) treatment is employed, in addition or possibly in place of filtering, to overcome infrastructural limitations on water throughput, such as filtering and/or pumping capacity. UV may be used over longer periods and/or with respect to higher throughputs, to reduce bacterial growth rates more significantly than filtering, and thereby prevent bacterial growth in the reservoir. UV treatment may be applied according to a pre-defined temporal pattern selected to prevent biomass growth in the reservoir and optionally be updated with respect to water quality measurements.

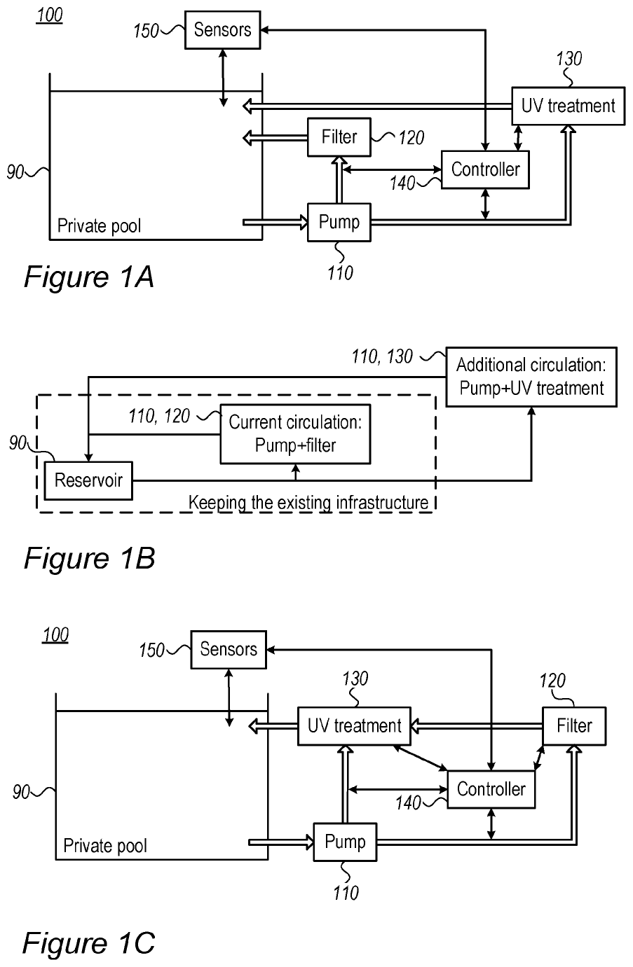

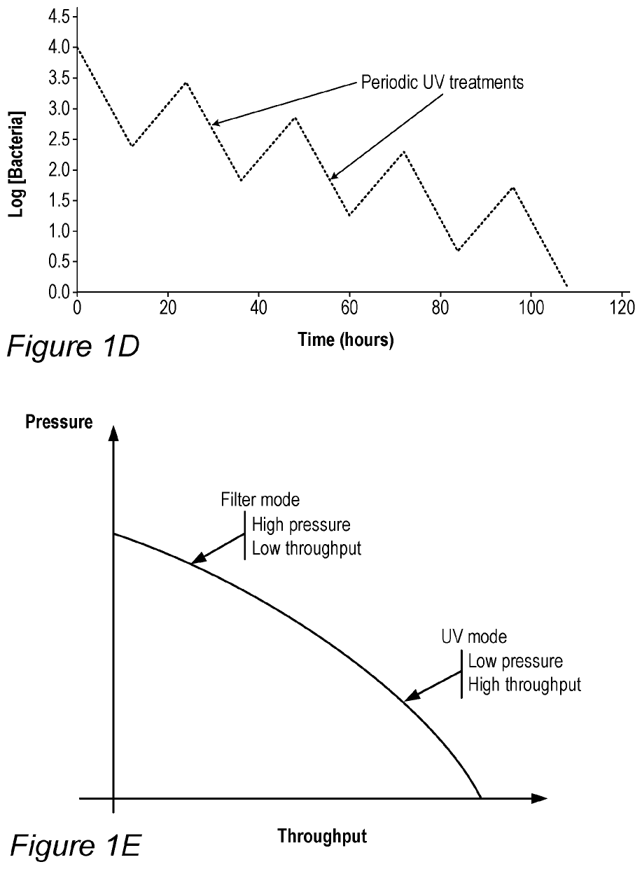

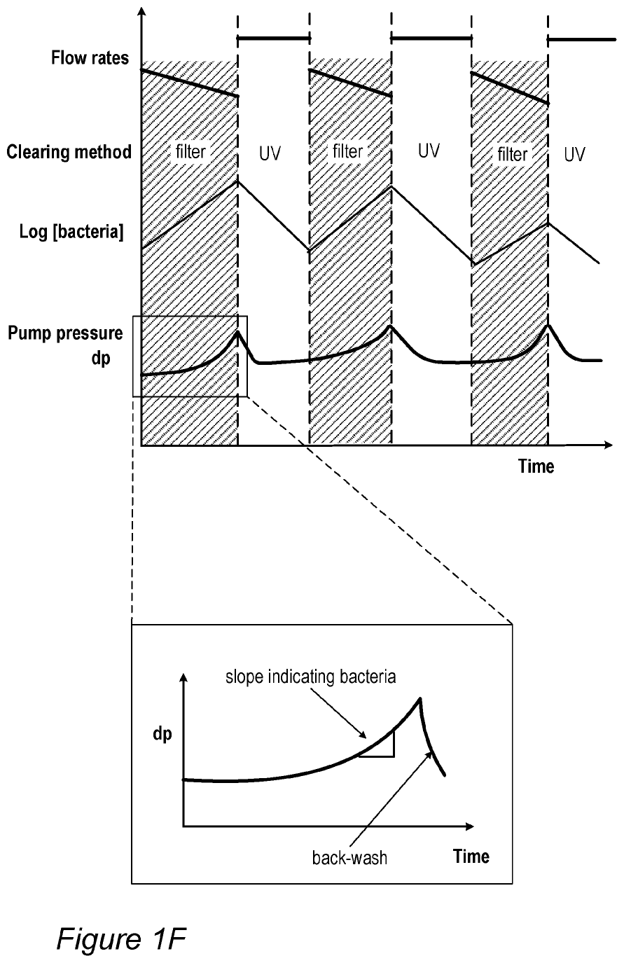

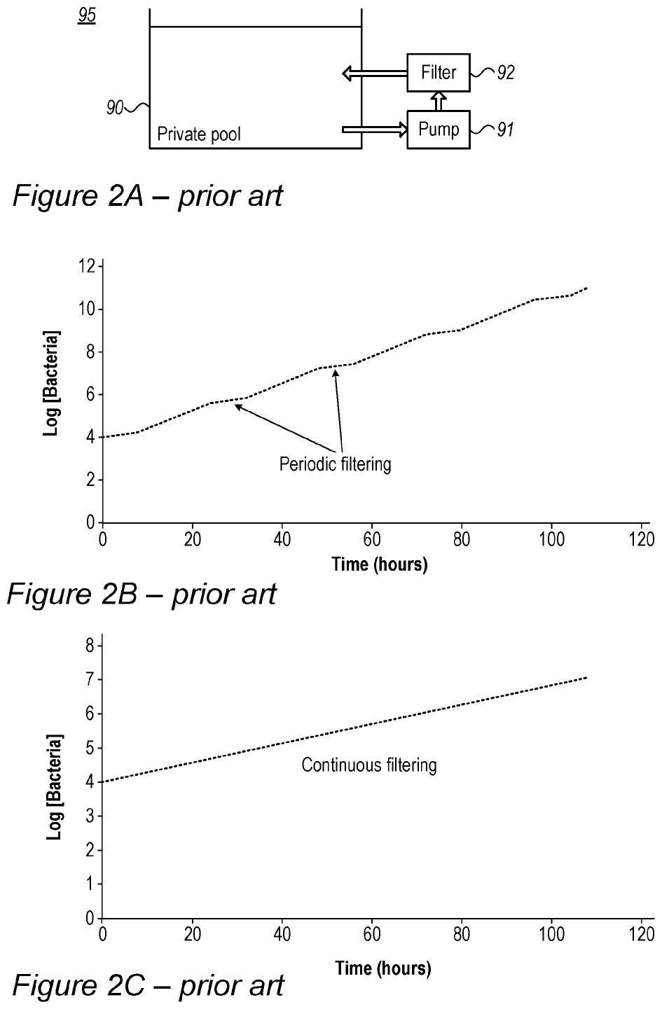

FIGS. 1A-1C are high-level schematic illustrations of a water treatment system 100, according to some embodiments of the invention. FIG. 1D provides simulation results depicting the expected bacterial concentration over time using water treatment system 100, according to some embodiments of the invention. As contrast, FIG. 2A is a high-level schematic illustration of prior art water treatment 95 of private pools 90. FIGS. 2B and 2C provide simulation results depicting the expected bacterial concentration over time for two filtering durations in the prior art.

In the prior art, small water reservoirs such as private pools 90 are usually not treated with any chemicals (other than large reservoirs, e.g., public pools), but water treatment 95 with a small pump 91 and a small filter 92 is applied occasionally to remove organic material and suspended solids from the water (as illustrated schematically in FIG. 2A). However, as noted by the inventors, prior art water treatment 95 is not sufficient to keep the water safe without addition of chemicals. For example, the simulation results presented in FIGS. 2B and 2C indicate that under operation of water treatment 95 during 8 or even 24 hours a day, respectively, the latter representing unrealistic constant cycling of the pool water through filter 92, germ concentration keeps rising, basically due to its exponential growth rate (as illustrated schematically in FIGS. 2B and 2C). The model assumptions refer to Escherichia coli (E. coli), assuming a generation doubling time of 3.5 hours which was determined experimentally (using 21 samples of filtered dechlorinated public pool water, sampled periodically over six days), a pool water volume of 30 m³ and a throughput of pump 91 and filter 92 of 4 m³/hour, which is typical for water treatment 95 of small reservoirs. Disclosed embodiments may be applicable to a range of volumes of small reservoirs 90 and to a range of throughputs of their limited infrastructure. For example, the typical volume of a private pool may be 90 m³, or between 50-200 m³ and typical infrastructure throughput may be 10 m³/hour or between 5 and 20 m³/hour. As clearly visible in the simulation results, even continuous operation of water treatment 95 is not sufficient to prevent contamination of small reservoirs such as private pools, mainly due to the limitations of not using chemicals and the relatively small throughput of pump 91 and filter 92, resulting from the typical limitations related to costs and infrastructure for private pools.

Disclosed water treatment systems 100, for treating reservoirs 90 without addition of chemicals, comprise a filtering unit 120, an ultraviolet (UV) treatment unit 130, a pumping unit 110 configured to circulate water from reservoir 90 through filtering unit 120 and back to reservoir 90, and configured to circulate water from reservoir 90 through UV treatment unit 130 and back to reservoir 90. Water treatment systems 100 further comprise a control unit 140 configured to operate pumping unit 110, filtering unit 120 and UV treatment unit 130 at a pre-defined temporal pattern selected to prevent biomass growth in reservoir 90, as illustrated schematically in FIGS. 1A-1C. FIG. 1A illustrates schematically embodiments with filtering unit 120 and UV treatment unit 130 configured as parallel units after pumping unit 110, FIG. 1B illustrates schematically embodiments with two parallel circuits, one with filtering unit 120 and another with UV treatment unit 130, using one or more pumping unit 110 in system 100 (e.g., one pumping unit 110 for each circuit or a common pumping unit 110 for both circuits), and FIG. 1C illustrates schematically embodiments in which water from reservoir 90 may either be pumped through UV treatment unit 130 and returned to reservoir 90 or be pumped through filtering unit 120 first and then and through UV treatment unit 130—with filtering unit 120 and UV treatment unit 130 being operable in series, or UV treatment unit 130 may be operable without pumping unit 110, and using the same infrastructure, e.g., same pumping unit 110. It is noted that the addition of UV treatment unit 130 allows preventing water contamination while keeping the same reservoir infrastructure and without addition of chemicals.

Examples for UV treatment include a radiation intensity between 10 and 40 mJ/cm² by Low pressure type lamps, medium pressure UV lamps and/or UV LED (light emitting diodes) lamps.

Schematic simulation results, assuming a pool water volume of 30 m³, a throughput of pumping unit 110 and UV treatment unit 130 of 16 m³/hour, for 12 hours a day and without filtering, assuming ultraviolet water transmission (UVT) of 90% and UV illumination by 300-1000W UV system, indicate effective reduction in the simulated E. coli population, reaching elimination of contamination within three days, as illustrated schematically in FIG. 1D. Compared with FIGS. 2B and 2C, that indicate the inability of prior art filtering systems to prevent water contamination (as bacterial concentrations starting from 10⁴ continue to rise in spite of repetitive and even continuous filtering)—disclosed embodiments illustrate the possibility to mitigate and prevent water contamination, reducing bacterial concentration from 10⁴ to zero within a few operation cycles—as presented in FIG. 1D. The periodic UV treatments were found to be able to reduce the bacterial growth rate effectively, eliminating contamination even without operation of the filtering unit.

Advantageously, UV water treatment is more effective than filtering because UV treatment can handle much larger throughputs than filtering units, under domestic operation conditions and limitations. Accordingly adding UV treatment to filters 92 may allow preventing contamination of small reservoirs such as private pools.

In certain embodiments, the water circulation through filtering unit 120 and the water circulation through UV treatment unit 130 may be parallel and independent of each other. In certain embodiments, the water circulation through filtering unit 120 may be limited to 8 hours a day, while the water circulation through UV treatment unit 130 may be carried out during at least 12 hours a day and/or according to requirements from water monitoring. For example, water treatment system 100 may further comprise a sensor unit 150 configured to sense a biomass level in the water. Control unit 140 may be further configured to receive biomass level measurement from sensor unit 150 and update the temporal pattern if the measured biomass level exceeds a pre-defined threshold.

For example, FIGS. 1E and 1F are high-level schematic illustrations of pump operation according to water contamination level, according to some embodiments of the invention. FIG. 1E provides a schematic operation curve for pumping unit 110, typically enabling high pressure pumping at small throughput and low pressure pumping at large throughput. The former operation profile is typically used for filtering by filtering unit 120 while the latter operation profile may be used for UV treatment by UV treatment unit 130. Such operation may be implemented, e.g., in parallel arrangement of filtering unit 120 and UV treatment unit 130 illustrated schematically in FIG. 1B, or possibly in partially parallel arrangement of filtering unit 120 and UV treatment unit 130 illustrated schematically in FIG. 1C (with the small filtered throughput also being UV treated, while UV treatment can be carried without operating filtering unit 120—thus enabling large throughput). FIG. 1F illustrates schematically (using arbitrary units) the operation of filtering and UV treatment cycles, e.g., by controller 140, with respect to pumping resistance at pumping unit 110, independently or in addition to sensor input. FIG. 1F illustrates schematically the rise in required pumping pressure dp as the amount to suspended particles, and hence filter resistance, increases, and the related increase in the bacterial contamination—during the filtering periods. Controller 140 may be configured to detect the rise in pumping pressure and switch system 100 to UV treatment mode—to reduce the bacterial concentration in reservoir 90. As bacterial contamination decreases, so does the amount of suspended particles, and consequently the filter resistance and the required pumping pressure. The increase in the pumping pressure may also be used as a signal for back washing filtering unit 120—to reduce its resistance. It is noted that in disclosed embodiments, filtering unit 120 and UV treatment unit 130 may be used to handle different aspects of reservoir contamination, with UV treatment unit 130 primarily treating microorganisms in the water and filtering unit 120 primarily removing suspended solids (that may result from various source, including bacterial contamination). Flow rates are high during the UV treatment, and decreasing during the filtering as the required pump pressure increases.

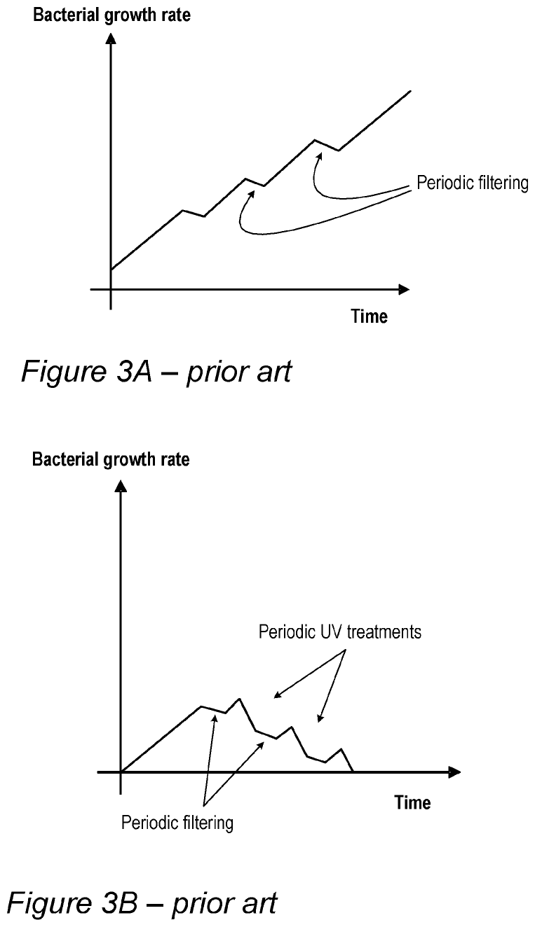

FIG. 3B is a high-level schematic illustration of the efficiency of UV water treatment, according to some embodiments of the invention, as contrasted with prior art use of filtering alone, illustrated schematically in FIG. 3A. Both graphs indicate schematically the bacterial growth rate over time, with periodic filtering and UV treatment. As illustrated in FIG. 3A, prior art periodic filtering mitigates bacterial growth, but is unable to prevent long term bacterial growth in the reservoir (as indicated experimentally in FIGS. 2B and 2C as well). In contrast, UV treatment can reduce bacterial growth more efficiently, and over longer periods of time than filtering, as it is less limited by existing infrastructure and especially by the size of the filter—and is able to handle larger water throughput (as indicated experimentally in FIG. 1D as well). The duration and throughput of UV treatment unit 130 may be adjusted to counter natural bacterial growth rates in any specific reservoir—by setting a pre-defined temporal pattern of UV treatment (e.g., the number of hours and/or water throughput of UV treatment per day) that provide sufficient reduction of bacterial growth. It is noted that UV treatment may be carried out over longer durations per day with smaller throughput, or over shorter durations per day with larger throughput—depending on existing infrastructure, e.g., on the pumping throughput. Specifically, UV treatment may be provided to reach a negative bacterial growth rate-preventing water contamination in the reservoir.

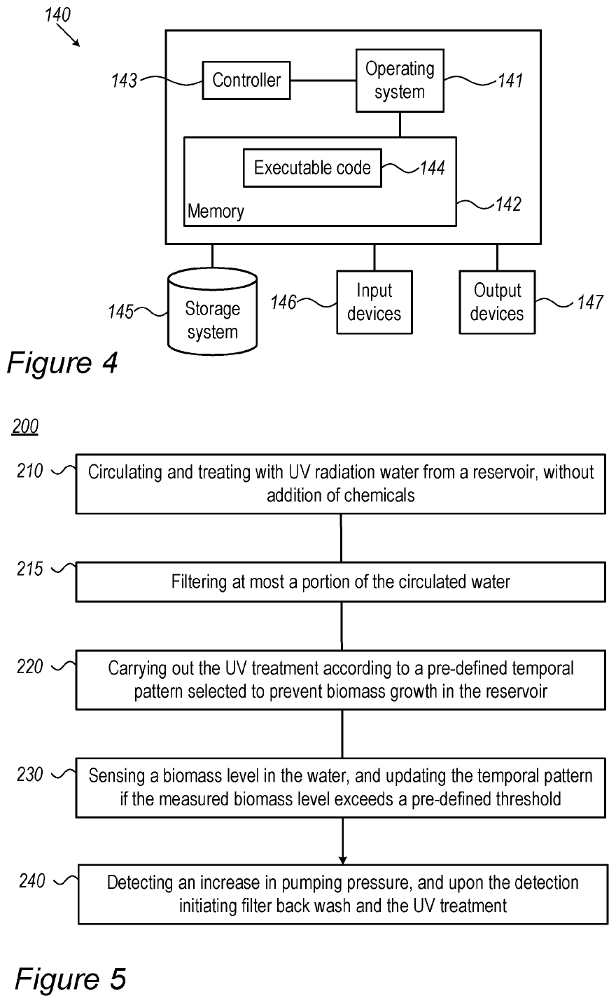

FIG. 4 is a high-level block diagram of exemplary controllers 140, which may be used with embodiments of the present invention. Controller(s) 140 may include one or more controller or processor 143 that may be or include, for example, one or more central processing unit processor(s) (CPU), one or more Graphics Processing Unit(s) (GPU or general-purpose GPU—GPGPU), a chip or any suitable computing or computational device, an operating system 141, a memory 142, a storage 145, input devices 146 and output devices 147.

Operating system 141 may be or may include any code segment designed and/or configured to perform tasks involving coordination, scheduling, arbitration, supervising, controlling, or otherwise managing operation of controller(s) 140, for example, scheduling execution of programs. Memory 142 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 142 may be or may include a plurality of possibly different memory units. Memory 142 may store for example, instructions to carry out a method (e.g., code 144), and/or data such as user responses, interruptions, etc.

Executable code 144 may be any executable code, e.g., an application, a program, a process, task or script. Executable code 144 may be executed by controller 143 possibly under control of operating system 141. For example, executable code 144 may when executed cause the production or compilation of computer code, or application execution such as VR execution or inference, according to embodiments of the present invention. Executable code 144 may be code produced by methods described herein. For the various modules and functions described herein, one or more computing devices and/or components of controller(s) 140 may be used. Devices that include components similar or different to those included in controller(s) 140 may be used and may be connected to a network and used as a system. One or more processor(s) 143 may be configured to carry out embodiments of the present invention by for example executing software or code.

Storage 145 may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, VR model data, parameters, etc. may be stored in a storage 145 and may be loaded from storage 145 into a memory 142 where it may be processed by controller 143. In some embodiments, some of the components shown in FIG. 4 may be omitted.

Input devices 146 may be or may include for example a mouse, a keyboard, a touch screen or pad or any suitable input device. It will be recognized that any suitable number of input devices may be operatively connected to controller(s) 140 as shown by block 146. Output devices 147 may include one or more displays, speakers and/or any other suitable output devices. It will be recognized that any suitable number of output devices may be operatively connected to controller(s) 140 as shown by block 147. Any applicable input/output (I/O) devices may be connected to controller(s) 140, for example, a wired or wireless network interface card (NIC), a modem, printer or facsimile machine, a universal serial bus (USB) device or external hard drive may be included in input devices 146 and/or output devices 147.

Embodiments of the invention may include one or more article(s) (e.g., memory 142 or storage 145) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

Certain embodiments comprise a computer program product (e.g., being part of controller 140) comprising a non-transitory computer readable storage medium having computer readable program embodied therewith, the computer readable program comprising computer readable program configured to receive measurements of a biomass level in water of a reservoir that is treated by ultraviolet (UV) radiation without addition of chemicals, and update a pre-defined temporal pattern of the UV treatment if the measured biomass level exceeds a pre-defined threshold.

FIG. 5 is a high-level flowchart illustrating a water treatment method 200 of treating a reservoir without addition of chemicals, according to some embodiments of the invention. The method stages may be carried out with respect to system water treatment systems 100 described above, which may optionally be configured to implement method 200. Method 200 may be at least partially implemented by at least one computer processor, e.g., in controller 140. Certain embodiments comprise computer program products comprising a computer readable storage medium having computer readable program embodied therewith and configured to carry out the relevant stages of method 200. Method 200 may comprise the following stages, irrespective of their order.

Method 200 may comprise circulating and treating with ultraviolet (UV) radiation water from the reservoir (stage 210), in addition to filtering at most a portion of the circulated water (stage 215), wherein the UV water treatment is carried out according to a pre-defined temporal pattern selected to prevent biomass growth in the reservoir (stage 220).

In some embodiments, UV treatment 210 may be carried out in parallel and independently of filtering 215. In some embodiments, UV treatment 210 may be carried out in series and in an additional path that circumvents filtering 215. For example, the water circulation through filtering 215 and UV treatment 210 may be serial (in series) and additional water circulation may be provided only through UV treatment 210, circumventing filtering 215—e.g., to increase the water treatment throughput without changing the infrastructure (e.g., circumventing throughput limitations of filtering 215). It is noted that UV may be used to treat large water throughputs (flows) that filtering with limited infrastructure cannot. Accordingly, UV treatment may be used complementarily to filtering both with respect to the type of treated contamination (filtering treating mainly suspended solids while UV treating mainly microorganisms) and with respect to the amount of treated water-both with respect to the existing level of contamination and available pumping capacity (see also FIGS. 1E and 1F and the related disclosure).

Filtering 215 may be carried out for at most two thirds of the circulated water, for a third or less of the circulated water or for intermediate throughputs.

Method 200 may further comprise sensing a biomass level in the water, and updating the temporal pattern if the measured biomass level exceeds a pre-defined threshold (stage 230). Method 200 may further comprise detecting an increase in pumping pressure (see, e.g., FIG. 1F), and upon the detection initiating filter back wash and the UV treatment (stage 240). The detection of the rise in pumping pressure may be used to trigger switching the operation mode from filtering to UV treatment.

Elements from FIGS. 1A-1F and 3B-5 may be combined in any operable combination, and the illustration of certain elements in certain figures and not in others merely serves an explanatory purpose and is non-limiting.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram or portions thereof.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams may represent a module, segment, or portion of code, which comprises one or more executable instructions for implementing the specified logical function(s). It should also be noted that, in some alternative implementations, the functions noted in the portion may occur out of the order noted in the figures. For example, two portions shown in succession may, in fact, be executed substantially concurrently, or the portions may sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A water treatment system for treating a reservoir without addition of chemicals, the water treatment system comprising:
a filtering unit,
an ultraviolet (UV) treatment unit,
a pumping unit configured to circulate water from the reservoir through the filtering unit and back to the reservoir, and configured to circulate water from the reservoir through the UV treatment unit and back to the reservoir, and
a control unit configured to operate the pumping unit, the filtering unit and the UV treatment unit at a pre-defined temporal pattern selected to prevent biomass growth in the reservoir.

2. The water treatment system of claim 1, wherein the water circulation through the filtering unit and the water circulation through the UV treatment unit are parallel and independent of each other.

3. The water treatment system of claim 2, wherein the water circulation through the filtering unit is limited to 8 hours daily, and the water circulation through the UV treatment unit is carried out during at least 12 hours daily.

4. The water treatment system of claim 1, wherein the water circulation through the filtering unit and the water circulation through the UV treatment unit are in series and additional water circulation is provided only through the UV treatment unit.

5. The water treatment system of claim 1, further comprising a sensor unit configured to sense a biomass level in the water, and wherein the control unit is further configured to receive biomass level measurement from the sensor unit and update the temporal pattern if the measured biomass level exceeds a pre-defined threshold.

6. The water treatment system of claim 1, wherein the control unit is further configured to detect an increase in pumping pressure of the pumping unit, and upon the detection initiate back washing the filtering unit and activating the UV treatment unit.

7. A water treatment method of treating a reservoir without addition of chemicals, the water treatment method comprising:
circulating and treating with ultraviolet (UV) radiation water from the reservoir, in addition to filtering at most a portion of the circulated water,
wherein the UV water treatment is carried out according to a pre-defined temporal pattern selected to prevent biomass growth in the reservoir.

8. The water treatment method of claim 7, wherein the filtering is carried out for at most two thirds of the circulated water.

9. The water treatment method of claim 7, wherein the filtering is carried out for a third or less of the circulated water.

10. The water treatment method of claim 7, wherein the UV treatment is carried out in parallel and independently of the filtering.

11. The water treatment method of claim 7, wherein the UV treatment is carried out in series and in an additional path that circumvents the filtering.

12. The water treatment method of claim 7, further comprising sensing a biomass level in the water, and updating the temporal pattern if the measured biomass level exceeds a pre-defined threshold.

13. The water treatment method of claim 7, further comprising detecting an increase in pumping pressure, and upon the detection initiating filter back wash and the UV treatment.

14. A computer program product comprising a non-transitory computer readable storage medium having computer readable program embodied therewith, the computer readable program comprising:
computer readable program configured to receive measurements of a biomass level in water of a reservoir that is treated by ultraviolet (UV) radiation without addition of chemicals, and update a pre-defined temporal pattern of the UV treatment if the measured biomass level exceeds a pre-defined threshold.

15. The computer program product of claim 14, further comprising computer readable program configured to detect an increase in pumping pressure, and upon the detection initiate filter back wash and the UV treatment.
